(19) United States
(12) Patent Application Publication
(10) Pub. No.: US 2026/0345577 A1
(43) Pub. Date: Nov. 5, 2026

(54) PHARMACEUTICAL COMPOSITION FOR PREVENTING OR TREATING PSORIASIS AND ATOPIC DERMATITIS

(71) Applicant: Byeung Mo CHANG, Seoul (KR)

(72) Inventor: Byeung Mo CHANG, Seoul (KR)

(21) Appl. No.: 19/669,938

(22) Filed: May 6, 2026

Related U.S. Application Data
(63) Continuation of application No. PCT/KR2024/017129, filed on Nov. 4, 2024.

(30) Foreign Application Priority Data
Nov. 7, 2023 (KR) 10-2023-0152789

(51) Int. Cl. A61K 31/4545; A61K 31/421; A61K 31/4409; A61K 47/10; A61K 47/12; A61K 47/14; A61K 47/18; A61K 47/20; A61K 47/22; A61P 31/04

(52) U.S. Cl. CPC A61K 31/4545; A61K 31/421; A61K 31/4409; A61K 47/10; A61K 47/12; A61K 47/14; A61K 47/18; A61K 47/20; A61K 47/22; A61P 31/04

(57) ABSTRACT

Disclosed is a pharmaceutical composition for preventing or treating psoriasis and atopic dermatitis. The pharmaceutical composition for preventing or treating psoriasis and atopic according to one embodiment includes: a 5 dermatitis phosphodiesterase-4 inhibitor including at least one of a phosphodiesterase-4 inhibitory component and a pharmaceutically acceptable salt thereof; and an active ingredient including at least any one of octenidine and an octenidine salt, wherein the phosphodiesterase-4 inhibitory component may include at least any one among roflumilast, difamilast, and tanimilast.

### CROSS-REFERENCE TO RELATED APPLICATION(S)

This application is continuation application of PCT/KR2024/017129 filed on Nov. 4, 2024, which claims priority to and the benefit of Korean Patent Application No. 10-2023-0152789 filed on Nov. 7, 2023, the disclosure of which is incorporated herein by reference in its entirety.

### BACKGROUND

The present disclosure relates to a pharmaceutical composition for preventing or treating psoriasis and atopic dermatitis.

Representative examples of skin diseases with a significant increase in patients due to industrial development and climate change include psoriasis and atopic dermatitis, both of which belong to systemic skin diseases. Currently, although many therapies for the treatment of psoriasis and atopic dermatitis are being applied, their therapeutic efficiency remains significantly low, and therefore, patients are suffering severe pain due to unbearable itching and skin inflammation.

Psoriasis involves symptoms where the skin thickens due to excessive keratin production on the skin surface along with erythematous symptoms of the skin, and is non-contagious but one of the chronic autoimmune skin diseases. That is, psoriasis is a chronic autoimmune disease that occurs when the immune system, which should attack microorganisms or irritants that have penetrated into the skin from the outside, attacks its own skin. According to what is known to date, molecular biological mediators inducing excessive immune responses include inflammatory cytokines, such as tumor necrosis factor-alpha (TNF-α), interleukin-17A (IL-17A), interleukin-23 (IL-23), and interleukin-33 (IL-33). Among these, the interleukin-17A and interleukin-23 have the greatest influence on psoriasis.

Drugs for the topical treatment of psoriasis (e.g., treatment using topical agents) include vitamin D derivatives, steroids, combined agents of vitamin D derivatives and steroids, and the like, which primarily act to promote normal cell differentiation, inhibit keratinocyte proliferation, and control anti-inflammatory and pruritus.

Meanwhile, atopic dermatitis affects 15-20% of children and 1-3% of adults worldwide and is a chronic, intractable inflammatory skin disease showing skin redness, lichenification, skin infections, and the like, along with pruritus. In skin where atopic dermatitis has occurred, T helper 2 (Th2), Th22, and Th17 cells, which are various subtypes of T cells, increase, and damage to the skin barrier is caused.

In addition, in skin where atopic dermatitis has occurred, thymic stromal lymphopoietin (TSLP) is secreted and activates Th2 cells to release interleukin-4 (IL-4), interleukin-5 (IL-5), interleukin-13 (IL-13), and interleukin-31 (IL-31). Among these, interleukin-4 and interleukin-13 have the greatest influence on atopic dermatitis.

Drugs for the topical treatment of atopic dermatitis include calcineurin inhibitors (e.g., tacrolimus, pimecrolimus, etc.), steroids (e.g., betamethasone, desonide, etc.), antihistamines (e.g., hydroxyzine, diphenhydramine, etc.), antibacterial agents (e.g., azithromycin, mupirocin, etc.), and the like.

In addition, microorganisms inducing inflammatory skin diseases include Staphylococcus aureus, Candida albicans, Malassezia sympodialis, and the like.

However, numerous lesion phenomena appearing in drugs and therapies for the treatment of psoriasis and atopic dermatitis (e.g., skin atrophy, growth retardation, malignant tumors, lymphopenia, vomiting, abdominal pain, nephrotoxicity, osteoporosis, avascular necrosis, hypertension, headache, bacterial and viral infections, etc.) are being widely reported as side effects.

Therefore, there is an urgent need for a therapeutic agent for psoriasis and atopic dermatitis that suppresses itching, prevents infection caused by various pathogens, restores the damaged skin barrier well, and has no side effects.

As a result of extensive efforts to develop a pharmaceutical composition that excellently kills microorganisms in the inflammatory affected areas of psoriasis and atopic dermatitis, improves the stability of pharmaceutical formulations used for psoriasis and atopic dermatitis, and further enhances the inhibitory activity against inflammatory cytokines inducing psoriasis and atopic dermatitis, the present inventors have completed the present invention.

### PRIOR ART DOCUMENTS

### Non-Patent Documents

(Non-Patent Document 1) 1. Young-Ae LEE, “Psoriasis and Dry Skin”, BRIC View 2020-T03, Jan. 9, 2020.
(Non-Patent Document 2) 2. Seong-Gyun GAM, “Key Players in Atopic Dermatitis and Development of New Drug,” Korea Pharmacist Journal, May 21, 2020.

### SUMMARY

In order to solve the aforementioned problems, the technical idea of the present disclosure provides a technology for a pharmaceutical composition with an excellent therapeutic effect on systemic skin diseases.

In addition, the technical idea of the present disclosure provides a technology for a pharmaceutical composition with excellent microbicidal effects against microorganisms inducing diseases such as psoriasis and atopic dermatitis.

Furthermore, the technical idea of the present disclosure provides a technology for a pharmaceutical composition with improved storage stability.

In addition, the technical idea of the present disclosure provides a technology for a pharmaceutical composition with an excellent inhibitory effect on inflammatory cytokines inducing psoriasis and atopic dermatitis.

In addition, the technical idea of the present disclosure provides a pharmaceutical composition for preventing or treating psoriasis.

Furthermore, the technical idea of the present disclosure provides a pharmaceutical composition for preventing or treating atopic dermatitis.

In addition, the technical idea of the present disclosure provides a use for the prevention or treatment of psoriasis and atopic dermatitis.

In addition, the technical idea of the present disclosure provides a method for preventing or treating psoriasis by applying a pharmaceutical composition to an affected area.

In addition, the technical idea of the present disclosure provides a method for preventing or treating atopic dermatitis by applying a pharmaceutical composition to an affected area.

The problems to be solved by the present invention are not limited to the aforementioned problems, and other technical problems not mentioned will be clearly understood by those skilled in the art to which the present invention pertains, from the following description.

In order to achieve these objects, in an embodiment of the present invention, the pharmaceutical composition for preventing or treating psoriasis and atopic dermatitis may comprise: a phosphodiesterase-4 inhibitor (PDE-4 inhibitor) comprising at least any one of a phosphodiesterase-4 (PDE-4) inhibitory component and a pharmaceutically acceptable salt thereof; and an active ingredient comprising at least any one of octenidine and an octenidine salt, wherein the phosphodiesterase-4 inhibitory component may comprise at least any one of roflumilast, difamilast, and tanimilast.

In addition, the phosphodiesterase-4 inhibitor may have a content of 0.01 to 5% by weight based on the total weight of the composition.

In addition, the phosphodiesterase-4 inhibitory component may further comprise at least any one of roflumilast N-oxide, lotamilast, apremilast, orismilast, crisaborole, and rolipram.

Furthermore, the pharmaceutically acceptable salt of the phosphodiesterase-4 inhibitory component may include at least any one selected from an inorganic ion salt, an inorganic acid salt, an organic acid salt, a sulfonic acid salt, an amino acid salt, and an amine salt of the phosphodiesterase-4 inhibitory component.

In addition, the active ingredient may have a content of 0.01 to 8% by weight based on the total weight of the composition, and the octenidine salt may include at least any one selected from an inorganic ion salt, an inorganic acid salt, an organic acid salt, a sulfonic acid salt, an amino acid salt, and an amine salt of the octenidine.

In addition, the pharmaceutical composition for preventing or treating psoriasis and atopic dermatitis may further comprise a crystal growth inhibitor comprising isopentyldiol and propylene carbonate in a weight ratio of 2:1, and the crystal growth inhibitor may have a content of 1 to 30% by weight based on the total weight of the composition.

Furthermore, the pharmaceutical composition for preventing or treating psoriasis and atopic dermatitis may further comprise an additive including at least any one of a solvent, a humectant, a penetrant, a surfactant, an antioxidant, a chelating agent, a pH adjuster, and a viscosity modifier.

In addition, the pharmaceutical composition for preventing or treating psoriasis and atopic dermatitis may have a pH of 2 to 10.

In addition, the pharmaceutical composition for preventing or treating psoriasis and atopic dermatitis may have a density of 0.1 to 2 g/cm³.

In addition, the pharmaceutical composition for preventing or treating psoriasis and atopic dermatitis may have a surface tension of 1 to 75 dyn/cm.

Furthermore, the pharmaceutical composition for preventing or treating psoriasis and atopic dermatitis may have a moisture content of 0.1 to 95% by weight.

In addition, the pharmaceutical composition for preventing or treating psoriasis and atopic dermatitis may have any one formulation selected from a solution, an ointment, a lotion, a cream, a gel, an emulsion, a suspension, a stick, a plaster, a patch, a cataplasma, a microcapsule, a cleaning agent, a liposomal drug, and a spray.

Meanwhile, another embodiment of the present invention may include a use for preparing a drug for the prevention or treatment of psoriasis and atopic dermatitis.

The means for solving the aforementioned problems are merely illustrative and should not be interpreted as intended to limit the present invention. In addition to the illustrative embodiments described above, additional embodiments described in the drawings and the detailed description of the invention may be present.

As described above, according to various embodiments of the present invention, it not only has excellent preventive and therapeutic effects on psoriasis and atopic dermatitis but also has high medication compliance due to its excellent microbicidal effects. That is, according to various embodiments of the present invention, it exhibits superior inhibitory activity against inflammatory cytokines compared to single-component formulations of PDE-4 inhibitors, and the stability of the pharmaceutical composition is excellent.

In addition, according to various embodiments of the present invention, the composition of the present invention exhibits much superior microbicidal effects compared to conventional single-component formulations of PDE-4 inhibitors. That is, the pharmaceutical composition according to various embodiments of the present invention has excellent microbicidal effects against Gram-positive bacteria (e.g., Staphylococcus aureus), yeasts, fungi, and the like.

In addition, according to various embodiments of the present invention, it preserves formulations in good condition, exhibits excellent microbicidal effects on inflammatory areas, and demonstrates improved inhibitory activity against inflammatory cytokines. That is, the pharmaceutical composition according to various embodiments of the present invention has improved microbicidal effects compared to conventional single-component formulations of PDE-4 inhibitors, thereby resulting in superior therapeutic effects for skin diseases, and may significantly improve storage stability of formulations compared to conventional single formulations containing only PDE-4 inhibitory components.

Furthermore, according to various embodiments of the present invention, the pharmaceutical composition has excellent storage stability even when stored at 45 °C for 4 weeks.

The effects according to various embodiments of the present invention are not limited to the aforementioned effects, and other effects not mentioned will be clearly understood by those skilled in the art from the description of the claims.

### BRIEF DESCRIPTION OF THE DRAWINGS

FIG. 1 is a graph showing the experimental results of the inhibitory activity against IL-17A and IL-23 according to Comparative Example 1 and Preparative Example 1.

FIG. 2 is a graph showing the experimental results of the inhibitory activity against IL-4 and IL-13 according to Comparative Example 1 and Preparative Example 1.

### DETAILED DESCRIPTION

Preferred embodiments of the present invention will be described in more detail with reference to the accompanying drawings, provided that well-known technical parts will be omitted or condensed for brevity of description.

In this specification, it should be noted that references to “an” or “one” embodiment of the present invention do not necessarily refer to the same embodiment, but mean at least one.

In the following embodiments, the singular expressions include the plural expressions unless the context clearly indicates otherwise.

In the following embodiments, the terms such as “comprise” or “have” mean that the features or components described in the specification are present, and do not exclude in advance the possibility of adding one or more other features or components.

In this specification, the term “prevention” refers to all acts of inhibiting or delaying the occurrence, spread, or recurrence of a disease by the administration of the pharmaceutical composition according to various embodiments of the present invention.

In this specification, the term “treatment” refers to all acts of improving or beneficially altering the symptoms of a disease by the administration of the pharmaceutical composition according to various embodiments of the present invention.

In this specification, the term “administration” refers to providing an active ingredient to a subject by any appropriate method.

In this specification, the term “subject” includes, but is not limited to, mammals including humans, guinea pigs, monkeys, cows, horses, sheep, pigs, chickens, turkeys, quails, cats, dogs, mice, rats, or rabbits, and preferably may be humans.

In this the term “pharmaceutically specification, acceptable” means that it is physiologically acceptable and does not typically cause allergic reactions, such as urticaria, gastrointestinal disorders, or dizziness, or similar reactions thereto, when administered to a human.

In this specification, the term “salt” refers to an acid addition salt formed by a pharmaceutically acceptable free acid. Therefore, the pharmaceutically acceptable salt refers to a salt commonly used in the pharmaceutical industry, and include, for example, inorganic ion salts prepared with calcium, potassium, sodium, magnesium, or the like; inorganic acid salts prepared with hydrochloric acid, nitric acid, phosphoric acid, hydrobromic acid, iodic acid, perchloric acid, sulfuric acid, or the like; organic acid salts prepared with acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, or the like; sulfonic acid salts prepared with methanesulfonic acid, ethanesulfonic acid, benzenesulfonic acid, salicylic acid, p-toluenesulfonic acid, naphthalenesulfonic acid, or the like; amino acid salts prepared with glycine, arginine, lysine, or the like; amine salts prepared from trimethylamine, triethylamine, ammonia, pyridine, picoline, or the like; and the like, but the types of salts referred to in this specification are not limited by these enumerated salts.

In this specification, the term “density” refers to a value representing mass per unit volume and is generally expressed in units of g/cm³. The density described in this specification can be measured by a tensiometer (Easy Dyne tensiometer model K20) sold by KRÜSS company.

In this specification, the term “moisture content” refers to the content of moisture contained in a pharmaceutical composition expressed as a percentage. The moisture content described in this specification is measured by the Karl Fischer titration method, and a Metrohm moisture meter (Metrohm 901 KF Titrando) may be used for the measurement.

In this specification, the term “surface tension” is generally expressed in units of dyn/cm, and refers to the force required to increase the unit area of a liquid surface or to increase the unit area of an interface between two liquids or between a liquid and a gas. The surface tension described in this specification is measured by the Du Nouy ring method using a tensiometer (Easy Dyne tensiometer model K20) sold by KRÜSS company.

The pharmaceutical composition for preventing or treating psoriasis and atopic dermatitis according to an embodiment may comprise a phosphodiesterase-4 inhibitor and an active ingredient. In an embodiment, the phosphodiesterase-4 inhibitor may comprise at least any one of a phosphodiesterase-4 inhibitory component and a pharmaceutically acceptable salt thereof.

According to an embodiment, the phosphodiesterase-4 inhibitory component may include at least any one of roflumilast, difamilast, tanimilast, roflumilast N-oxide, lotamilast, apremilast, orismilast, crisaborole, and rolipram.

In an embodiment, the roflumilast (CAS Registry Number: 162401-32-3) has the chemical name of 3-(cyclopropylmethoxy)-N-(3,5-dichloro-4-pyridinyl)-4-(difluoromethoxy)benzamide.

The roflumilast has a molecular formula of C₁₇H₁₄Cl₂F₂N₂O₃ (molecular weight: 403.2 g/mol, melting point: 158 °C), and is a white powder. The roflumilast may have a structural formula represented by following Formula 1:

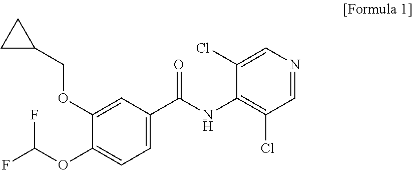 [SMILES: O=C(Nc1c(Cl)cncc1Cl)c1ccc(OC(F)F)c(OCC2CC2)c1]

The roflumilast is an inhibitory component that inhibits phosphodiesterase-4, and cyclic increases intracellular adenosine monophosphate (CAMP) levels in immune cells, endothelial cells, and keratinocytes to downregulate the expression of inflammatory cytokines, thereby being effective in treating psoriasis, which is an inflammatory skin disease.

In an embodiment, the pharmaceutically acceptable salt of the phosphodiesterase-4 inhibitory component may include at least any one selected from an inorganic ion salt, an inorganic acid salt, an organic acid salt, a sulfonic acid salt, an amino acid salt, and an amine salt of the phosphodiesterase-4 inhibitory component. For example, when the phosphodiesterase-4 inhibitory component is roflumilast, the roflumilast salt may include at least any one selected from an inorganic ion salt of the roflumilast, an inorganic acid salt of the roflumilast, an organic acid salt of the roflumilast, a sulfonic acid salt of the roflumilast, an amino acid salt of the roflumilast, and an amine salt of the roflumilast.

In an embodiment, the phosphodiesterase-4 inhibitor may have a content of 0.01 to 5% by weight based on the total weight of the composition. As a specific example, the phosphodiesterase-4 inhibitor may have a content of 0.01% by weight, 0.02% by weight, 0.03% by weight, 0.04% by weight, 0.05% by weight, 0.06% by weight, 0.07% by weight, 0.08% by weight, 0.09% by weight, 0.1% by weight, 0.2% by weight, 0.3% by weight, 0.4% by weight, 0.5% by weight, 0.6% by weight, 0.7% by weight, 0.8% by weight, 0.9% by weight, 18 by weight, 1.18 by weight, 1.2% by weight, 1.3% by weight, 1.4% by weight, 1.5% by weight, 1.6% by weight, 1.7% by weight, 1.8% by weight, 1.9% by weight, 2% by weight, 2.1% by weight, 2.2% by weight, 2.3% by weight, 2.4% by weight, 2.5% by weight, 2.6% by weight, 2.7% by weight, 2.8% by weight, 2.9% by weight, 3% by weight, 3.1% by weight, 3.2% by weight, 3.3% by weight, 3.4% by weight, 3.5% by weight, 3.6% by weight, 3.7% by weight, 3.8% by weight, 3.9% by weight, 4% by weight, 4.1% by weight, 4.2% by weight, 4.3% by weight, 4.4% by weight, 4.5% by weight, 4.6% by weight, 4.7% by weight, 4.8% by weight, 4.9% by weight, or 5% by weight. In addition, the phosphodiesterase-4 inhibitor may have a content in the range of one or more of the aforementioned values and one or less of the aforementioned values.

For example, the phosphodiesterase-4 inhibitor may have a content in the range of 0.01% by weight to 5% by weight, 0.01% by weight to 2% by weight, 0.05% by weight to 1.5% by weight, 0.1% by weight to 2% by weight, 0.5% by weight to 2% by weight, 0.01% by weight to 0.5% by weight, 0.5% by weight to 5% by weight, 0.01% by weight to 1% by weight, 1% by weight to 2% by weight, 1.5% by weight to 3% by weight, 2.5% by weight to 4% by weight, 3.5% by weight to 5% by weight, 4% by weight to 5% by weight, 1% by weight to 5% by weight, 2% by weight to 4% by weight, 1% by weight to 3% by weight, 3% by weight to 5% by weight, 0.01% by weight to 0.05% by weight, 0.1% by weight to 0.5% by weight, 0.5% by weight to 1% by weight, or 0.1% by weight to 3% by weight.

Furthermore, the phosphodiesterase-4 inhibitor may be applied in a content of one or more of the aforementioned values, or may be applied in a content of one or less of the aforementioned values. For example, the phosphodiesterase-4 inhibitor may be applied in a content of 0.01% by weight or more, 0.5% by weight or more, 18 by weight or more, 2% by weight or more, 3% by weight or more, or 4% by weight or more, or may be applied in a content of 5% by weight or less, 4% by weight or less, 3% by weight or less, 2% by weight or less, 1% by weight or less, 0.9% by weight or less, 0.8% by weight or less, 0.7% by weight or less, 0.6% by weight or less, 0.5% by weight or less, or 0.05% by weight or less.

In an embodiment, the content of the phosphodiesterase-4 inhibitor is not limited to the aforementioned examples and may be applied in an amount effective for the prevention or treatment of psoriasis and atopic dermatitis. The effective level for the prevention or treatment of psoriasis and atopic dermatitis may be determined depending on the factors including the type and severity of the patient's disease, the activity of the drug, the sensitivity to the drug, the time of administration, the route of administration, the rate of the excretion, the duration of treatment, and the drugs used simultaneously, and other factors well known in the medical field. Considering all of the aforementioned factors, it is important to administer an amount that can achieve the maximum effect in a minimal amount without side effects, which can be easily determined by those skilled in the art.

In an embodiment, the active ingredient may be at least any one of an antifungal ingredient and an antibacterial ingredient. For example, the active ingredient is an ingredient that exhibits microbicidal activity against Staphylococcus aureus, Candida albicans, Malassezia symphodialis, and the like. According to an embodiment, the active ingredient may include at least any one of octenidine and octenidine salt.

In an embodiment, the octenidine (CAS Registry Number: 71251-02-0) may have a structural formula represented by following Formula 2. The octenidine has a molecular formula of C₃₆H₆₂N₄ and a molecular weight of 550.9. The octenidine has antimicrobial activity and is, therefore, effective in killing bacteria and fungi.

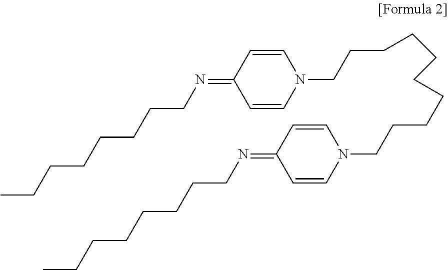 [SMILES: CCCCCCCCN=c1ccn(CCCCCCCCCCn2ccc(=NCCCCCCCC)cc2)cc1]

In an embodiment, the octenidine salt may include at least any one selected from an inorganic ion salt, an inorganic acid salt, an organic acid salt, a sulfonic acid salt, an amino acid salt, and salt of an amine the octenidine, as the pharmaceutically acceptable salt of the octenidine.

For example, the pharmaceutically acceptable salt of the octenidine includes inorganic ion salts prepared with calcium, potassium, sodium, magnesium, or the like; inorganic acid salts prepared with hydrochloric acid, nitric acid, phosphoric acid, hydrobromic acid, iodic acid, perchloric acid, sulfuric acid, or the like; organic acid salts prepared with acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, or the like; sulfonic acid salts prepared with methanesulfonic acid, ethanesulfonic acid, benzenesulfonic acid, salicylic acid, p-toluenesulfonic acid, naphthalenesulfonic acid, or the like; amino acid salts prepared with glycine, arginine, lysine, or the like; amine salts prepared from trimethylamine, triethylamine, ammonia, pyridine, picoline, or the like; and the like, but the types of octenidine salts are not limited by these enumerated salts.

As a specific example, the pharmaceutically acceptable salt of the octenidine may be applied as octenidine dihydrochloride. The octenidine dihydrochloride (CAS Registry Number: 70775-75-6) is a white or slightly yellowish powder and is a pyridine-based compound.

The octenidine dihydrochloride has the chemical name of N-octyl-1-[10-(4-octyliminopyridin-1-yl) decyl]pyridin-4-imine dihydrochloride. The octenidine dihydrochloride has a molecular formula of C₃₆H₆₄Cl₂N₄ (molecular weight: 623.84, melting point: 215-217 °C). The octenidine dihydrochloride may have a structural formula represented by following Formula 3.

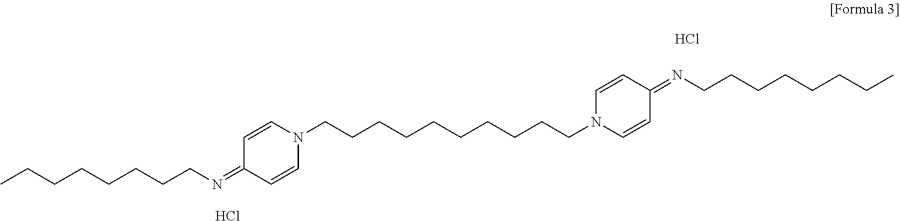 [SMILES: Cl ; CCCCCCCCN=c1ccn(CCCCCCCCCCn2ccc(=NCCCCCCCC)cc2)cc1 ; Cl]

In an embodiment, the octenidine dihydrochloride strongly adsorbs to bacterial cell surfaces presenting anions, binding to phospholipids and anionic polysaccharides of the cell membrane, and degrading cellular functions to cause plasma membrane leakage and inhibit mitochondrial function, thereby exhibiting excellent microbicidal effects. In addition, the octenidine dihydrochloride inhibits the activation of Langerhans cells and suppresses the secretion of inflammatory cytokines, thereby demonstrating excellent anti-inflammatory activity.

Psoriasis is an inflammatory skin disease caused by the expression of inflammatory cytokines (e.g., TNF-α, IL-17A, IL-23, IL-33, etc.), which are molecular biological mediators inducing an excessive immune response; and among these, IL-17A and IL-23 have the most significant influence. Main causative pathogens of infections occurring due to psoriasis include Staphylococcus aureus, Candida albicans, and the like.

Atopic dermatitis is an inflammatory skin disease caused by the release of IL-4, IL-5, IL-13, IL-31, and the like from Th2 cells activated by the secretion of thymic stromal lymphopoietin; and among these, IL-4 and IL-13 have the most significant influence. Main causative pathogens of infections occurring due to atopic dermatitis include Staphylococcus aureus, Malassezia sympodialis, and the like.

The pharmaceutical composition for preventing or treating psoriasis and atopic dermatitis according to an embodiment may strongly kill bacteria and fungi through complex antimicrobial actions, thereby exhibiting a significantly superior antibiotic effect compared to single formulations containing only PDE-4 inhibitory components, and can excellently improve the effects of preventing and treating inflammatory skin diseases.

In an embodiment, the active ingredient may have a content of 0.01 to 8% by weight based on the total weight of the composition. As a specific example, the active ingredient may have a content of 0.01% by weight, 0.02% by weight, 0.03% by weight, 0.04% by weight, 0.05% by weight, 0.06% by weight, 0.07% by weight, 0.08% by weight, 0.09% by weight, 0.1% by weight, 0.2% by weight, 0.3% by weight, 0.4% by weight, 0.5% by weight, 0.6% by weight, 0.7% by weight, 0.8% by weight, 0.9% by weight, 1% by weight, 1.1% by weight, 1.2% by weight, 1.3% by weight, 1.4% by weight, 1.5% by weight, 1.6% by weight, 1.7% by weight, 1.8% by weight, 1.9% by weight, 2% by weight, 2.1% by weight, 2.2% by weight, 2.3% by weight, 2.4% by weight, 2.5% by weight, 2.6% by weight, 2.7% by weight, 2.8% by weight, 2.9% by weight, 3% by weight, 3.1% by weight, 3.2% by weight, 3.3% by weight, 3.4% by weight, 3.5% by weight, 3.6% by weight, 3.7% by weight, 3.8% by weight, 3.9% by weight, 4% by weight, 4.1% by weight, 4.2% by weight, 4.3% by weight, 4.4% by weight, 4.5% by weight, 4.6% by weight, 4.7% by weight, 4.8% by weight, 4.9% by weight, 5% by weight, 5.1% by weight, 5.2% by weight, 5.3% by weight, 5.4% by weight, 5.5% by weight, 5.6% by weight, 5.7% by weight, 5.8% by weight, 5.9% by weight, 6% by weight, 6.18 by weight, 6.2% by weight, 6.3% by weight, 6.4% by weight, 6.5% by weight, 6.6% by weight, 6.7% by weight, 6.8% by weight, 6.9% by weight, 7% by weight, 7.1% by weight, 7.2% by weight, 7.3% by weight, 7.4% by weight, 7.5% by weight, 7.6% by weight, 7.7% by weight, 7.8% by weight, 7.9% by weight, or 8% by weight. In addition, the active ingredient may have a content in the range of one or more of the aforementioned values and one or less of the aforementioned values.

For example, the active ingredient may have a content in the range of 0.01% by weight to 3% by weight, 0.05% by weight to 2.5% by weight, 0.1% by weight to 3% by weight, 0.5% by weight to 2.5% by weight, 1% by weight to 2% by weight, 1% by weight to 3% by weight, 2% by weight to 4% by weight, 3% by weight to 5% by weight, 4% by weight to 6% by weight, 5% by weight to 7% by weight, 6% by weight to 8% by weight, or 0.01% by weight to 8% by weight.

Furthermore, the active ingredient may be applied in a content of one or more of the aforementioned values, or may be applied in a content of one or less of the aforementioned values. For example, the active ingredient may be applied in a content of 0.01% by weight or more, 0.1% by weight or more, 0.5% by weight or more, 1% by weight or more, 2% by weight or more, 3% by weight or more, 4% by weight or more, 5% by weight or more, 6% by weight or more, or 7% by weight or more, or may be applied in a content of 8% by weight or less, 7% by weight or less, 6% by weight or less, 5% by weight or less, 4% by weight or less, 3% by weight or less, 2% by weight or less, 1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, or 0.05% by weight or less. In an embodiment, the content of the active ingredient is not limited only to the aforementioned examples and may be applied in an amount effective for the prevention or treatment of psoriasis and atopic dermatitis.

The pharmaceutical composition for preventing or treating psoriasis and atopic dermatitis according to an embodiment may further comprise a crystal growth inhibitor. In an embodiment, the crystal growth inhibitor may inhibit the crystallization of specific components contained in the pharmaceutical composition. For example, the crystal growth inhibitor may prevent a phenomenon in which at least any one of the phosphodiesterase-4 inhibitor and the active ingredient contained in the composition is precipitated as crystals.

In an embodiment, the crystal growth inhibitor may have a content of 1 to 30% by weight based on the total weight of the composition. As a specific example, the crystal growth inhibitor may have a content of 1% by weight, 1.5% by weight, 2% by weight, 2.5% by weight, 3% by weight, 3.5% by weight, 4% by weight, 4.5% by weight, 5% by weight, 5.5% by weight, 6% by weight, 6.5% by weight, 7% by weight, 7.5% by weight, 8% by weight, 8.5% by weight, 9% by weight, 9.5% by weight, 10% by weight, 10.5% by weight, 11% by weight, 11.5% by weight, 12% by weight, 12.5% by weight, 13% by weight, 13.5% by weight, 14% by weight, 14.5% by weight, 15% by weight, 15.5% by weight, 16% by weight, 16.5% by weight, 17% by weight, 17.5% by weight, 18% by weight, 18.5% by weight, 19% by weight, 19.5% by weight, 20% by weight, 20.5% by weight, 21% by weight, 21.5% by weight, 22% by weight, 22.5% by weight, 23% by weight, 23.5% by weight, 24% by weight, 24.5% by weight, 25% by weight, 25.5% by weight, 26% by weight, 26.5% by weight, 27% by weight, 27.5% by weight, 28% by weight, 28.5% by weight, 29% by weight, 29.5% by weight, or 30% by weight. In addition, the crystal growth inhibitor may have a content in the range of one or more of the aforementioned values and one or less of the aforementioned values.

For example, the crystal growth inhibitor may have a content in the range of 18 by weight to 30% by weight, 1% by weight to 25% by weight, 1% by weight to 20% by weight, 1% by weight to 10% by weight, 10% by weight to 30% by weight, 10% by weight to 20% by weight, 5% by weight to 20% by weight, 5% by weight to 30% by weight, 15% by weight to 30% by weight, 15% by weight to 20% by weight, 20% by weight to 30% by weight, 25% by weight to 30% by weight, 5% by weight to 10% by weight, 5% by weight to 15% by weight, 1% by weight to 5% by weight, or 12% by weight to 30% by weight.

In addition, the crystal growth inhibitor may be applied in a content of one or more of the aforementioned values, or may be applied in a content of one or less of the aforementioned values. For example, the crystal growth inhibitor may be applied in a content of 1% by weight or more, 2% by weight or more, 3% by weight or more, 4% by weight or more, 5% by weight or more, 6% by weight or more, 7% by weight or more, 8% by weight or more, 9% by weight or more, 10% by weight or more, 15% by weight or more, or 20% by weight or more, or may be applied in a content of 30% by weight or less, 25% by weight or less, 20% by weight or less, 15% by weight or less, or 10% by weight or less.

According to an embodiment, the crystal growth inhibitor may include isopentyldiol and propylene carbonate. In an embodiment, the isopentyldiol to the propylene carbonate may be a weight ratio of 2:1. If the weight ratio of isopentyldiol to propylene carbonate deviates from 2:1, the storage stability of the composition may decrease, and crystals may precipitate or precipitates may form during storage of the composition, and there is a concern that the physical properties of the composition may be adversely affected.

In the pharmaceutical composition according to an embodiment, the isopentyldiol and the propylene carbonate in a weight ratio of 2:1 may be used as the crystal growth inhibitor during low-temperature (e.g., −15 °C) storage, in an amount of 1 to 30% by weight based on the total weight of the composition. The pharmaceutical composition for preventing or treating psoriasis and atopic dermatitis according to an embodiment may further comprise an additive. In an embodiment, the additive may include at least any one of a solvent, a humectant, a penetrant, a surfactant, an antioxidant, a chelating agent, a pH adjuster, and a viscosity modifier.

In an embodiment, the solvent may include at least any one of an organic solvent, water for injection (WFI), purified water, and physiological saline. The organic solvent may include at least any one selected from the group consisting of alkanes, alkenes, alcohols, esters, ketones, and ethers. For example, the organic solvent may include at least any one of ethanol, isopropyl alcohol, dimethyl sulfoxide, acetone, and ethyl acetate. According to an embodiment, ethanol, isopropyl alcohol, dimethyl sulfoxide, purified water, and the like may be used as the solvent. In an embodiment, the solvent may have a content of 1% by weight or more to 90% by weight or less (e.g., 1% by weight, 5% by weight, 10% by weight, 15% by weight, 20% by weight, 25% by weight, 30% by weight, 35% by weight, 40% by weight, 45% by weight, 50% by weight, 55% by weight, 60% by weight, 65% by weight, 70% by weight, 75% by weight, 80% by weight, 85% by weight, or 90% by weight) based on the total weight of the composition.

In an embodiment, the humectant is an additive that provides moisture to the stratum corneum of the skin or prevents excessive skin dryness. The humectant according to an embodiment may include at least any one selected from the group consisting of isoamyl laurate, propylene glycol, glycerin, polyethylene glycol 400, sorbitol, hyaluronic acid, and mixtures thereof. Preferably, isoamyl laurate, polyethylene glycol 400, hyaluronic acid, and the like may be used as the humectant. In an embodiment, the humectant may have a content of 18 by weight or more to 60% by weight or less (e.g., 1% by weight, 5% by weight, 10% by weight, 15% by weight, 20% by weight, 25% by weight, 30% by weight, 35% by weight, 40% by weight, 45% by weight, 50% by weight, 55% by weight, or 60% by weight) based on the total weight of the composition.

In an embodiment, the penetrant is a substance that is added to enhance therapeutic effects by increasing the penetration of a drug into the skin. The penetrant according to an embodiment may include at least any one selected from the group consisting of diisopropyl adipate, diethyl sebacate, oleyl oleate, capric/caprylic triglyceride, 2-methyl-1,3-propanediol, and mixtures thereof. Preferably, diisopropyl adipate, oleyl oleate, capric/caprylic triglyceride, and the like may be used as the penetrant. In an embodiment, the penetrant may have a content of 1% by weight or more to 80% by weight or less (e.g., 1% by weight, 5% by weight, 10% by weight, 15% by weight, 20% by weight, 25% by weight, 30% by weight, 35% by weight, 40% by weight, 45% by weight, 50% by weight, 55% by weight, 60% by weight, 65% by weight, 70% by weight, 75% by weight, or 80% by weight) based on the total weight of the composition.

In an embodiment, the surfactant may include at least any one selected from the group consisting of Poloxamer 407, Poloxamer 188, sorbitan monolaurate, sorbitan monooleate, sorbitan trioleate, polyglycerol-10-laurate, polysorbate 80, and mixtures Poloxamer 407, sorbitan thereof. Preferably, trioleate, polyglycerol-10-laurate, polysorbate 80, and the like may be used as the surfactant. In an embodiment, the surfactant may have a content of 1% by weight or more to 30% by weight or less (e.g., 1% by weight, 5% by weight, 10% by weight, 15% by weight, 20% by weight, 25% by weight, or 30% by weight) based on the total weight of the composition.

In an embodiment, the antioxidant may include at least any one selected from the group consisting of butylhydroxytoluene (BHT), butylhydroxyanisole (BHA), ascorbic acid, tocopherol, ferulic acid, propyl gallate, cysteine, sodium sulfite, and mixtures thereof. Preferably, butylhydroxytoluene, ascorbic acid, tocopherol, cysteine, and the like may be used as the antioxidant. In an embodiment, the antioxidant may have a content of 0.001% by weight or more to 3% by weight or less (e.g., 0.001% by weight, 0.005% by weight, 0.01% by weight, 0.05% by weight, 0.1% by weight, 0.5% by weight, 1% by weight, 1.5% by weight, 2% by weight, 2.5% by weight, or 3% by weight) based on the total weight of the composition.

In an embodiment, the chelating agent is a sequestering agent that binds with oxygen, nitrogen, or various metal ions to inactivate them. The chelating agent according to an embodiment may include at least any one selected from the group consisting of pentasodium pentetate, glutamic acid, methylglycine, disodium ethylenediaminetetraacetic acid (disodium EDTA), tetrasodium ethylenediaminetetraacetic acid (tetrasodium EDTA), and mixtures thereof. Preferably, pentasodium pentetate, disodium ethylenediaminetetraacetic acid, tetrasodium ethylenediaminetetraacetic acid, and the like may be used as the chelating agent. In an embodiment, the chelating agent may have a content of 0.001% by weight or more to 3% by weight or less (e.g., 0.001% by weight, 0.005% by weight, 0.01% by weight, 0.05% by weight, 0.1% by weight, 0.5% by weight, 1% by weight, 1.5% by weight, 2% by weight, 2.5% by weight, or 3% by weight) based on the total weight of the composition.

In an embodiment, the pH adjuster may include at least any one selected from the group consisting of hydrochloric acid, phosphoric acid, pentetic acid, acetic acid, citric acid, succinic acid, lactic acid, malic acid, tartaric acid, triethanolamine, sodium hydroxide, sodium citrate, calcium citrate, diisopropylamine, and mixtures thereof. Preferably, the pH of the composition may be adjusted to a range of 2 or more to 10 or less (e.g., 2, 3, 4, 5, 6, 7, 8, 9, or 10) using pentetic acid, citric acid, tartaric acid, sodium hydroxide, or sodium citrate. In an embodiment, the pH adjuster may have a content of 0.01% by weight or more to 3% by weight or less (e.g., 0.01% by weight, 0.05% by weight, 0.1% by weight, 0.5% by weight, 1% by weight, 1.5% by weight, 2% by weight, 2.5% by weight, or 3% by weight) based on the total weight of the composition.

In an embodiment, the viscosity modifier may include at least any one selected from the group consisting of sodium alginate, carboxymethyl cellulose, sodium polyacrylate, pectin, gelatin, hydroxypropyl methylcellulose, dextrin, povidone, and mixtures thereof. Preferably, carboxymethyl cellulose, sodium polyacrylate, hydroxypropyl methyl cellulose, povidone, and the like may be used as viscosity modifiers. Depending on the viscosity of the pharmaceutical composition, the viscosity modifier may or may not be added to the composition.

The additive for the pharmaceutical composition according to an embodiment may include ingredients commonly used in the art, and examples are listed in the literature (Raymond C. Rowe. Handbook of Pharmaceutical Excipients 7th edition). In addition, the aforementioned types of additives, such as solvents, humectants, penetrants, surfactants, antioxidants, chelating agents, pH adjusters, and viscosity modifiers, may be prepared by adding them in various compositions according to the intended use and conditions, and if necessary, the use of at least some of the types of additives may be omitted, or other known types of components may be additionally used.

In an embodiment, the pharmaceutical composition for preventing or treating psoriasis and atopic dermatitis may be implemented in the form of a solution comprising 0.01 to 5% by weight of roflumilast, 0.01 to 8% by weight of octenidine dihydrochloride, 1 to 90% by weight of a solvent (e.g., ethanol, dimethyl sulfoxide), 1 to 60% by weight of a humectant (e.g., polyethylene glycol 400), 1 to 80% by weight of a penetrant (e.g., diisopropyl adipate), 1 to 30% by weight of a surfactant, 0.001 to 3% by weight of an antioxidant, 0.001 to 3% by weight of a chelating agent, 0.01 to 3% by weight of a pH adjuster (e.g., citric acid), and 1 to 30% by weight of a crystal growth inhibitor (the weight ratio of isopentyl diol to propylene carbonate is 2:1).

In another embodiment, the pharmaceutical composition for preventing or treating psoriasis and atopic dermatitis may be implemented in the form of a solution comprising 0.01 to 5% by weight of difamilast, 0.01 to 8% by weight of octenidine dihydrochloride, 1 to 90% by weight of a solvent (e.g., ethanol, dimethyl sulfoxide), 1 to 60% by weight of a humectant (e.g., polyethylene glycol 400), 1 to 80% by weight of a penetrant (e.g., diisopropyl adipate), 1 to 30% by weight of a surfactant, 0.001 to 3% by weight of an antioxidant, 0.001 to 3% by weight of a chelating agent, 0.01 to 3% by weight of a pH adjuster (e.g., citric acid), and 1 to 30% by weight of a crystal growth inhibitor (the weight ratio of isopentyl diol to propylene carbonate is 2:1).

In another embodiment, the pharmaceutical composition for preventing or treating psoriasis and atopic dermatitis may be implemented in the form of a solution comprising 0.01 to 5% by weight of tanimilast, 0.01 to 8% by weight of octenidine dihydrochloride, 1 to 90% by weight of a solvent (e.g., ethanol, dimethyl sulfoxide), 1 to 60% by weight of a humectant (e.g., polyethylene glycol 400), 1 to 80% by weight of a penetrant (e.g., diisopropyl adipate), 1 to 30% by weight of a surfactant, 0.001 to 3% by weight of an antioxidant, 0.001 to 3% by weight of a chelating agent, 0.01 to 3% by weight of a pH adjuster (e.g., citric acid), and 1 to 30% by weight of a crystal growth inhibitor (the weight ratio of isopentyl diol to propylene carbonate is 2:1).

Meanwhile, the pharmaceutical composition for preventing or treating psoriasis and atopic dermatitis according to an embodiment may have a pH of 2 to 10. As a specific example, the pharmaceutical composition for preventing or treating psoriasis and atopic dermatitis may have a pH of 2, 2.5, 3, 3.5, 4, 4.5, 5, 5.5, 6, 6.5, 7, 7.5, 8, 8.5, 9, 9.5, or 10. In addition, the pharmaceutical composition for preventing or treating psoriasis and atopic dermatitis according to an embodiment may have a pH in the range of one or more of the aforementioned values and one or less of the aforementioned values. For example, the pharmaceutical composition may have a pH in the range of 2 to 10, 3 to 9, 4 to 8, 5 to 7, 4 to 9, 4 to 10, 5 to 9.5, 6 to 8, or 7 to 8.

The pharmaceutical composition for preventing or treating psoriasis and atopic dermatitis according to an embodiment may have a density of 0.1 to 2 g/cm³. As a specific example, the pharmaceutical composition for preventing or treating psoriasis and atopic dermatitis may be applied in a density of 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1 g/cm³, 1.1 g/cm³, 1.2 g/cm³, 1.3 g/cm³, 1.4 g/cm³, 1.5 g/cm³, 1.6 g/cm³, 1.7 g/cm³, 1.8 g/cm³, 1.9 g/cm³, or 2 g/cm³.

In addition, in an embodiment, the pharmaceutical composition for preventing or treating psoriasis and atopic dermatitis may have a density in the range of one or more of the aforementioned values and one or less of the aforementioned values. For example, the pharmaceutical composition may have a density in the range of 0.1 g/cm³ to 2 g/cm³, 0.4 g/cm³ to 1 g/cm³, 0.8 g/cm³ to 1.5 g/cm³, 1.2 g/cm³ to 1.7 g/cm³, 1 g/cm³ to 2 g/cm³, 1.5 g/cm³ to 2 g/cm³, 0.5 g/cm³ to 2 g/cm³, 0.8 g/cm³ to 2 g/cm³, 1.2 g/cm³ to 1.8 g/cm³, 1.5 g/cm³ to 1.7 g/cm³, or 1.5 g/cm³ to 2 g/cm³.

In an embodiment, the pharmaceutical composition for preventing or treating psoriasis and atopic dermatitis may have a surface tension of 1 to 75 dyn/cm. As a specific example, the pharmaceutical composition for preventing or treating psoriasis and atopic dermatitis may be applied in a surface tension of 1 dyn/cm, 2 dyn/cm, 3 dyn/cm, 4 dyn/cm, 5 dyn/cm, 6 dyn/cm, 7 dyn/cm, 8 dyn/cm, 9 dyn/cm, 10 dyn/cm, 11 dyn/cm, 12 dyn/cm, 13 dyn/cm, 14 dyn/cm, 15 dyn/cm, 16 dyn/cm, 17 dyn/cm, 18 dyn/cm, 19 dyn/cm, 20 dyn/cm, 21 dyn/cm, 22 dyn/cm, 23 dyn/cm, 24 dyn/cm, 25 dyn/cm, 26 dyn/cm, 27 dyn/cm, 28 dyn/cm, 29 dyn/cm, 30 dyn/cm, 31 dyn/cm, 32 dyn/cm, 33 dyn/cm, 34 dyn/cm, 35 dyn/cm, 36 dyn/cm, 37 dyn/cm, 38 dyn/cm, 39 dyn/cm, 40 dyn/cm, 41 dyn/cm, 42 dyn/cm, 43 dyn/cm, 44 dyn/cm, 45 dyn/cm, 46 dyn/cm, 47 dyn/cm, 48 dyn/cm, 49 dyn/cm, 50 dyn/cm, 51 dyn/cm, 52 dyn/cm, 53 dyn/cm, 54 dyn/cm, 55 dyn/cm, 56 dyn/cm, 57 dyn/cm, 58 dyn/cm, 59 dyn/cm, 60 dyn/cm, 61 dyn/cm, 62 dyn/cm, 63 dyn/cm, 64 dyn/cm, 65 dyn/cm, 66 dyn/cm, 67 dyn/cm, 68 dyn/cm, 69 dyn/cm, 70 dyn/cm, 71 dyn/cm, 72 dyn/cm, 73 dyn/cm, 74 dyn/cm, or 75 dyn/cm at 25 °C.

In addition, the pharmaceutical composition according to an embodiment may have a surface tension in the range of one or more of the aforementioned values and one or less of the aforementioned values. For example, the pharmaceutical composition may have a surface tension in the range of 1 dyn/cm to 75 dyn/cm, 5 dyn/cm to 60 dyn/cm, 10 dyn/cm to 50 dyn/cm, 15 dyn/cm to 65 dyn/cm, 20 dyn/cm to 60 dyn/cm, 10 dyn/cm to 40 dyn/cm, 15 dyn/cm to 30 dyn/cm, 25 dyn/cm to 55 dyn/cm, 30 dyn/cm to 50 dyn/cm, 41 dyn/cm to 50 dyn/cm, 42 dyn/cm to 50 dyn/cm, 43 dyn/cm to 50 dyn/cm, 44 dyn/cm to 50 dyn/cm, 45 dyn/cm to 50 dyn/cm, 41 dyn/cm to 45 dyn/cm, 41 dyn/cm to 44 dyn/cm, 41 dyn/cm to 43 dyn/cm, or 50 dyn/cm to 75 dyn/cm.

In an embodiment, the moisture content of the pharmaceutical composition for preventing or treating psoriasis and atopic dermatitis is measured by the Karl Fischer titration method, and the moisture content of the composition measured by the Karl Fischer titration method may be 0.1 to 95% by weight. As a specific example, the pharmaceutical composition for preventing or treating psoriasis and atopic dermatitis may be applied in a moisture content of 0.1%, 0.28, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%.

In addition, the pharmaceutical composition for preventing or treating psoriasis and atopic dermatitis may have a moisture content in the range of one or more of the aforementioned values and one or less of the aforementioned values. For example, the pharmaceutical composition may have a moisture content in the range of 0.1 to 95%, 0.5 to 90%, 0.1 to 80%, 1 to 70%, 2 to 5%, 3 to 58, 5 to 85%, 7 to 60%, 8 to 558, 9 to 50%, 10 to 45%, 10 to 20%, 10 to 15%, 10 to 13%, 15 to 40%, or 20 to 35%.

The pharmaceutical composition for preventing or treating psoriasis and atopic dermatitis according to an embodiment may be prepared in any one formulation selected from a solution, an ointment, a lotion, a cream, a gel, an emulsion, a suspension, a stick, a plaster, a patch, a cataplasma, a microcapsule, a cleaning agent, a liposomal drug, and a spray.

The pharmaceutical composition according to various embodiments of the present invention may be prepared by mixing the aforementioned components according to conventional methods, and since it does not undergo heating and cooling processes, the convenience and economic efficiency of process control may be improved. If necessary, solutions may be prepared, by adding a phosphodiesterase-4 inhibitor and an active ingredient according to the input ratio, and first preparing a stock solution using ethanol and dimethyl sulfoxide as solvents, and then additionally mixing other components.

Hereinafter, the present invention will be described in more detail through specific preparative examples. The following are preparative examples merely illustrative to aid understanding of the present invention, and therefore, the scope of rights of the present invention is not limited or restricted thereto.

### Preparation of Solutions According to Comparative Examples 1 to 3 and Preparative Examples 1 to 9

Solutions containing a phosphodiesterase-4 inhibitor and octenidine dihydrochloride were prepared according to each component and composition described in Tables 1, 2, and 3 below. The content of each component indicated in Tables 1 to 3 represents % by weight relative to the total solution. Transparent solutions were prepared, by charging ethanol and dimethyl sulfoxide into three preparation vessels at room temperature (25 °C), adding and dissolving a phosphodiesterase-4 inhibitory component (roflumilast, difamilast, tanimilast) in each preparation vessel, and then sequentially adding octenidine dihydrochloride, polyethylene glycol 400, diisopropyl adipate, isopentyldiol, propylene carbonate, Poloxamer 407, butylhydroxytoluene, disodium ethylenediaminetetraacetic acid, and citric acid to each preparation vessel, and adding purified water until the total weight of the solution reached 100% by weight, and then filtering them through a 0.45 μm filter. As a result of measuring the pH, density, surface tension, and moisture content of the prepared solution, the pH was 4 to 8, the density was 0.8 to 1 g/cm³, the surface tension (at 25 °C) was 35 to 55 dyn/cm, and the moisture content was 1 to 10% by weight.

TABLE 1

| | Preparative Examples (% by weight) | | | |
|---|---|---|---|---|
| | Comparative | Preparative | Preparative | Preparative |
| Components | Example 1 | Example 1 | Example 2 | Example 3 |
| Roflumilast | 0.50 | 0.50 | 0.01 | 5.00 |
| Octenidine dihydrochloride | — | 0.10 | 8.00 | 0.01 |
| Ethanol | 20.00 | 20.00 | 20.00 | 20.00 |
| Dimethyl sulfoxide | 20.00 | 20.00 | 20.00 | 20.00 |
| Polyethylene glycol 400 | 10.00 | 10.00 | 10.00 | 10.00 |
| Diisopropyl adipate | 10.00 | 10.00 | 10.00 | 10.00 |
| Isopentyl diol | 20.00 | 20.00 | 20.00 | 20.00 |
| Propylene carbonate | 10.00 | 10.00 | 10.00 | 10.00 |
| Poloxamer 407 | 1.00 | 1.00 | 1.00 | 1.00 |
| Butylhydroxytoluene | 0.01 | 0.01 | 0.01 | 0.01 |
| Disodium ethylenediamine | 0.01 | 0.01 | 0.01 | 0.01 |
| tetraacetic acid | | | | |
| Citric acid | 0.10 | 0.10 | 0.10 | 0.10 |
| Purified water | 8.38 | 8.28 | 0.87 | 3.87 |
| Total (% by weight) | 100.00 | 100.00 | 100.00 | 100.00 |

TABLE 2

| | Preparative Examples (% by weight) | | | |
|---|---|---|---|---|
| | Comparative | Preparative | Preparative | Preparative |
| Components | Example 2 | Example 4 | Example 5 | Example 6 |
| Difamilast | 0.50 | 0.50 | 0.01 | 5.00 |
| Octenidine dihydrochloride | — | 0.10 | 8.00 | 0.01 |
| Ethanol | 20.00 | 20.00 | 20.00 | 20.00 |
| Dimethyl sulfoxide | 20.00 | 20.00 | 20.00 | 20.00 |
| Polyethylene glycol 400 | 10.00 | 10.00 | 10.00 | 10.00 |
| Diisopropyl adipate | 10.00 | 10.00 | 10.00 | 10.00 |
| Isopentyl diol | 20.00 | 20.00 | 20.00 | 20.00 |
| Propylene carbonate | 10.00 | 10.00 | 10.00 | 10.00 |
| Poloxamer 407 | 1.00 | 1.00 | 1.00 | 1.00 |
| Butylhydroxytoluene | 0.01 | 0.01 | 0.01 | 0.01 |
| Disodium ethylenediamine | 0.01 | 0.01 | 0.01 | 0.01 |
| tetraacetic acid | | | | |
| Citric acid | 0.10 | 0.10 | 0.10 | 0.10 |
| Purified water | 8.38 | 8.28 | 0.87 | 3.87 |
| Total (% by weight) | 100.00 | 100.00 | 100.00 | 100.00 |

TABLE 3

| | Preparative Examples (% by weight) | | | |
|---|---|---|---|---|
| | Comparative | Preparative | Preparative | Preparative |
| Components | Example 3 | Example 7 | Example 8 | Example 9 |
| Tanimilast | 0.50 | 0.50 | 0.01 | 5.00 |
| Octenidine dihydrochloride | — | 0.10 | 8.00 | 0.01 |
| Ethanol | 20.00 | 20.00 | 20.00 | 20.00 |
| Dimethyl sulfoxide | 20.00 | 20.00 | 20.00 | 20.00 |
| Polyethylene glycol 400 | 10.00 | 10.00 | 10.00 | 10.00 |
| Diisopropyl adipate | 10.00 | 10.00 | 10.00 | 10.00 |
| Isopentyl diol | 20.00 | 20.00 | 20.00 | 20.00 |
| Propylene carbonate | 10.00 | 10.00 | 10.00 | 10.00 |
| Poloxamer 407 | 1.00 | 1.00 | 1.00 | 1.00 |
| Butylhydroxytoluene | 0.01 | 0.01 | 0.01 | 0.01 |
| Disodium ethylenediamine | 0.01 | 0.01 | 0.01 | 0.01 |
| tetraacetic acid | | | | |
| Citric acid | 0.10 | 0.10 | 0.10 | 0.10 |
| Purified water | 8.38 | 8.28 | 0.87 | 3.87 |
| Total (% by weight) | 100.00 | 100.00 | 100.00 | 100.00 |

### Test Example 1. Susceptibility Testing for the Evaluation of Antimicrobial Activity

In general, clinical therapeutic outcomes can be predicted through susceptibility testing of antimicrobial agents. The test strains used in the susceptibility testing for the evaluation of antimicrobial activity are Staphylococcus aureus, Candida albicans, and Malassezia sympodialis, which are causative pathogens inducing atopic dermatitis and psoriasis. This experiment was conducted using the tube dilution technique to evaluate the antimicrobial activity against the aforementioned three types of microorganisms. The results of comparing Comparative Examples 1 to 3 and Preparative Examples 1 to 9 as the minimum inhibition concentration (MIC₉₀) against each microorganism for antimicrobial activity evaluation are as described in Tables 4, 5, and 6 below. The minimum inhibition concentration test was performed in accordance with Clinical and Laboratory Standards Institute (CLSI) standards, using the CLSI M07-A10:2015 method for the Gram-positive bacterium Staphylococcus aureus, and the CLSI M27-A3:2008 method for the yeasts Candida albicans and Malassezia sympodialis, as follows:

### 1) Pre-Culture of Test Strains

\textcircled{1} Staphylococcus aureus: Inoculated onto Tryptic Soy Agar (TSA) medium and cultured at 35±2 °C for 16 to 24 hours.
\textcircled{2} Candida albicans and Malassezia sympodialis: Inoculated onto Sabouraud Dextrose Agar (SDA) medium and cultured at 35±2 °C for 20 to 24 hours.

### 2) Preparation of the Test Bacterial Solution and Spore Suspensions

\textcircled{1} Staphylococcus aureus: Diluted in Cation-Adjusted Muller-Hinton Broth (CAMHB) medium to a concentration of 1×10⁶ CFU/ml and used as the test microorganism solution.
\textcircled{2} Candida albicans and Malassezia symphodialis: RPMI 1640 medium (Gibco) was buffered with 0.165 mol/L 3-N-morpholinopropane sulfonic acid (MOPS), and then spore suspensions were prepared at concentrations of 5.0×10² to 2.5×10³ CFU/ml and used as the test microorganism solutions.

### 3) Inoculation of Test Microorganism Solutions

\textcircled{1} Staphylococcus aureus: 1 ml of the test microorganism solution was inoculated into each tube into which the test solutions of different concentrations had been dispensed. Tubes containing 2 ml of the solution (Comparative Examples 1 to 3, Preparative Examples 1 to 9) in CAMHB medium were used as negative controls, and tubes containing 1 ml of the solution (Comparative Examples 1 to 3, Preparative Examples 1 to 9) and 1 ml of the test microorganism solution in CAMHB medium were used as positive controls. The tubes treated with the test microorganism solution were incubated at 35±2 °C for 16 to 24 hours.
\textcircled{2} Candida albicans and Malassezia sympodialis: 0.9 ml of the test microorganism solution was inoculated into each well into which the test solutions of different concentrations had been dispensed. Wells containing 1.0 ml of the solution (Comparative Examples 1 to 3, Preparative Examples 1 to 9) in RPMI 1640 medium buffered with 0.165 mol/L MOPS were used as negative controls, and wells containing 0.1 ml of the solution (Comparative Examples 1 to 3, Preparative Examples 1 to 9) and 0.9 ml of the test microorganism solution in RPMI 1640 medium buffered with 0.165 mol/L MOPS were used as positive controls. The tubes treated with the test microorganism solution were incubated at 35±2 °C for 24 to 48 hours.

### 4) Determination of Results

After culture, the minimum concentration at which no microbial growth was visually observed in each tube was considered the minimum inhibition concentration. After culture, no growth must be observed in the negative controls, and microbial growth must be observed in the positive controls. The results obtained by the aforementioned antimicrobial activity test methods are described in Tables 4, 5, and 6.

TABLE 4

| | Minimum inhibition concentrations (MIC₉₀, μg/ml) | | | |
|---|---|---|---|---|
| | Comparative | Preparative | Preparative | Preparative |
| Strains | Example 1 | Example 1 | Example 2 | Example 3 |
| Staphylococcus aureus | 0.16 | 0.03 | 0.02 | 0.06 |
| (S. aureus) ATCC 6538 | | | | |
| Candida albicans (C. | 0.26 | 0.05 | 0.03 | 0.09 |
| albicans) ATCC 10231 | | | | |
| Malassezia sympodialis | 0.19 | 0.03 | 0.02 | 0.07 |
| (M. sympodialis) | | | | |
| ATCC 42132 | | | | |

TABLE 5

| | Minimum inhibition concentrations (MIC₉₀, μg/ml) | | | |
|---|---|---|---|---|
| | Comparative | Preparative | Preparative | Preparative |
| Strains | Example 2 | Example 4 | Example 5 | Example 6 |
| Staphylococcus aureus | 0.28 | 0.05 | 0.03 | 0.11 |
| (S. aureus) ATCC 6538 | | | | |
| Candida albicans (C. | 0.32 | 0.06 | 0.04 | 0.12 |
| albicans) ATCC 10231 | | | | |
| Malassezia sympodialis | 0.18 | 0.03 | 0.02 | 0.07 |
| (M. sympodialis) | | | | |
| ATCC 42132 | | | | |

TABLE 6

| | Minimum inhibition concentrations (MIC₉₀, μg/ml) | | | |
|---|---|---|---|---|
| | Comparative | Preparative | Preparative | Preparative |
| Strains | Example 3 | Example 7 | Example 8 | Example 9 |
| Staphylococcus aureus | 0.26 | 0.05 | 0.03 | 0.10 |
| (S. aureus) ATCC 6538 | | | | |
| Candida albicans (C. | 0.36 | 0.07 | 0.04 | 0.15 |
| albicans) ATCC 10231 | | | | |
| Malassezia sympodialis | 0.24 | 0.05 | 0.03 | 0.09 |
| (M. sympodialis) | | | | |
| ATCC 42132 | | | | |

As can be seen from the results of Tables 4, 5, and 6 above, the compositions according to Preparative Examples 1 to 9 showed increased antimicrobial activity against significantly Staphylococcus aureus, Candida albicans, and Malassezia sympodialis compared to Comparative Examples 1 to 3, which do dihydrochloride. The antimicrobial not contain octenidine activities of Preparative Examples 1 to 3 increased by 2.7 to 8 times against Staphylococcus aureus, 2.9 to 8.7 times against Candida albicans, and 2.7 to 9.5 times against Malassezia sympodialis, compared to those of Comparative Example 1. The Examples 4 to 6 antimicrobial activities of Preparative increased by 2.6 to 9.3 times against Staphylococcus aureus, 2.7 to 8 times against Candida albicans, and 2.6 to 9 times against Malassezia sympodialis, compared to those of Comparative Example 2. The antimicrobial activities of Preparative Examples 7 to 9 increased by 2.6 to 8.7 times against Staphylococcus aureus, 2.4 to 9 times against Candida albicans, and 2.7 to 8 times against Malassezia sympodialis, compared to those of Comparative Example 3. Therefore, the pharmaceutical compositions according to Preparative Examples 1 to 9 show good susceptibility testing results against causative pathogens inducing psoriasis and atopic dermatitis, and thus can be expected to show excellent clinical therapeutic outcomes.

### Test Example 2. Evaluation of Storage Stability

10 ml of each sample from Comparative Examples 1 to 3 and Preparative Examples 1 to 9 was placed into 20 ml vials, respectively, and hermetically sealed, and then stored at 45 °C for 4 weeks. The ultraviolet-visible absorption spectra of samples were measured at the initial stage and after 4 weeks at 45 °C. That is, the absorbance was measured at 500 nm using a spectrophotometer (Mega-800, SCINCO CO., LTD., Korea), and the results are described in Table 7 below.

TABLE 7
[Absorbance at 500 nm]

| Samples | Initial | After 4 weeks |
|---|---|---|
| Comparative Example 1 | 0.046 ± 0.0002 | 0.049 ± 0.0003 |
| Preparative Example 1 | 0.074 ± 0.0001 | 0.075 ± 0.0002 |
| Preparative Example 2 | 0.092 ± 0.0003 | 0.094 ± 0.0002 |
| Preparative Example 3 | 0.122 ± 0.0004 | 0.126 ± 0.0001 |
| Comparative Example 2 | 0.041 ± 0.0001 | 0.044 ± 0.0002 |
| Preparative Example 4 | 0.053 ± 0.0001 | 0.054 ± 0.0002 |
| Preparative Example 5 | 0.134 ± 0.0002 | 0.137 ± 0.0003 |
| Preparative Example 6 | 0.176 ± 0.0002 | 0.182 ± 0.0001 |
| Comparative Example 3 | 0.061 ± 0.0003 | 0.065 ± 0.0001 |
| Preparative Example 7 | 0.063 ± 0.0002 | 0.064 ± 0.0002 |
| Preparative Example 8 | 0.172 ± 0.0003 | 0.175 ± 0.0001 |
| Preparative Example 9 | 0.181 ± 0.0005 | 0.186 ± 0.0003 |

As described in Table 7, the solutions of Preparative Examples 1 to 9 showed improved stability compared to Comparative Examples 1 to 3, which do not contain octenidine dihydrochloride. In the absorbance measured at the 500 nm after 4 weeks, Comparative Example 1 showed an increase of about 7%, whereas Preparative Examples 1 to 3 showed an increase at a level of about 1 to 3%; Comparative Example 2 showed an increase of about 78, whereas Preparative Examples 4 to 6 showed an increase at a level of about 2 to 3%; and Comparative Example 3 showed an increase of about 7%, whereas Preparative Examples 7 to 9 showed an increase at a level of about 2 to 38. Therefore, it can be seen that the compositions of Preparative Examples 1 to 9 have superior stability compared to Comparative Examples 1 to 3, even after 4 weeks at 45 °C.

### Test Example 3. Evaluation of the Inhibitory Activity Against Inflammatory Cytokines

The inhibitory activities of Comparative Examples 1 to 3 and Preparative Examples 1 to 9 against inflammatory cytokines were evaluated by the method described below.

### (1) Cytotoxicity and Inhibitory Activity Against Inflammatory Cytokines in Keratinocytes

Keratinocytes (HaCaT) were purchased from the Korean Cell Line Bank (Seoul, Korea) and used. The keratinocytes were cultured in DMEM (Dulbecco's Modified Eagle Medium; Gibco BRL., USA) broth supplemented with 10% heat-inactivated fetal bovine serum (HIFBS; Gibco BRL, USA) and 1% penicillin/streptomycin (100 IU/ml penicillin, 100 μg/ml streptomycin; Thermo Fisher Scientific) for 24 hours in an incubator under conditions of 37 °C and 5% CO₂.

### \textcircled{1} Cytotoxicity Evaluation (MTT Assay)

100 μl of the cultured keratinocytes were seeded into a 96-well plate at 1×10⁶ cells/ml and cultured for 24 hours under conditions of 37 °C and 5% CO₂, and then the medium was removed, and 100 μl of DMEM medium and 100 μl of the samples (Comparative Examples 1 to 3, Preparative Examples 1 to 9) were added to each well and cultured for 24 hours. After 24 hours, 10 μl of 3-(4,5-dimethylthiazol-2-yl)-2,5-diphenyl tetrazolium bromide (MTT) solution (0.5 mg/ml, Sigma Aldrich, St. Louis, MO, USA) was added to each well and cultured for an additional 4 hours to reduce MTT, and then the supernatant was removed. Subsequently, 100 μl of dimethyl sulfoxide (DMSO) was dispensed into each well and mixed for 1 hour, and then the absorbance was measured at 540 nm using an enzyme-linked immunosorbent assay microplate reader (ELISA microplate reader; Bio-Rad model 680, Bio-Rad Laboratories INC., Tokyo, Japan). Cell viability was calculated as a percentage of the absorbance of the sample-treated group relative to the absorbance of the sample-untreated group, and the results are described in Table 8.

* Cell viability (%)=(Absorbance of sample-treated group/Absorbance of sample-untreated group)×100

### \textcircled{2} Inhibitory Activity Against Inflammatory Cytokines

The keratinocytes were seeded into a 96-well plate at a density of 5×10⁵ cells per well and cultured for 24 hours, and then the supernatant was washed with phosphate-buffered saline (PBS). To induce an immune response in the keratinocytes, samples prepared by placing 10 ng/ml of tumor necrosis factor-(TNF-α) and 10 ng/ml of interferon-γ (IFN-γ) into HIFBS-free DMEM medium and adding 100 μg/ml of each of Comparative Examples 1 to 3 and Preparative Examples 1 to 9 were added to each well and cultured for 24 hours, and then the concentrations of IL-17A and IL-23 in the supernatant of the keratinocytes were measured using an ELISA kit (MAX DELUXE; BioLegend, USA).

Briefly, ELISA plates were coated with 100 μl of diluted (1×) capture antibody and cultured at 4 °C for 24 hours, and then washed 4 times with washing buffer (0.05 v/v % Tween-20 in PBS), and then 200 μl of blocking buffer (3 w/v % bovine serum albumin in Tris-buffered saline, pH 7.6) was added and cultured at room temperature for 1 hour with shaking, and then washed 4 times with washing buffer. IL-17A and IL-23 standards were diluted to a concentration range of 0 to 1,000 μg/ml, and then added to the wells of the ELISA plates at 100 μl each, cultured at room temperature for 2 hours with shaking, and washed 4 times, and then 100 μl of detecting antibody was added and cultured at room temperature for 1 hour with shaking, and then washed 4 times. Subsequently, 100 μl of avidin-horseradish peroxidase (HRP) reagent (1:1,000) was added and cultured at room temperature for 30 minutes with shaking, and then washed 5 times. Finally, 100 μl of TMB (3,3′,5,5′-tetramethyl benzidine) substrate solution was added and cultured in the dark for 30 minutes, and then 100 μl of stop solution (2 N H₂SO₄) was added, the absorbance was measured at 450 nm within 15 minutes using a microplate reader (Bio Tek Instruments, Inc.; Vermont, USA), and each cytokine inhibition concentration is described in Table 8.

TABLE 8

| | | Cytokine inhibitory | |
|---|---|---|---|
| | Cytotoxicity | activity (pg/ml) | |
| Samples | (cell viability %) | IL-17A | IL-23 |
| Comparative Example 1 | 100 | 76.7 | 84.3 |
| Preparative Example 1 | 101 | 21.2 | 22.4 |
| Preparative Example 2 | 99 | 16.6 | 17.5 |
| Preparative Example 3 | 100 | 30.8 | 34.5 |
| Comparative Example 2 | 100 | 80.6 | 87.5 |
| Preparative Example 4 | 101 | 21.4 | 23.1 |
| Preparative Example 5 | 100 | 18.3 | 20.2 |
| Preparative Example 6 | 102 | 27.5 | 31.8 |
| Comparative Example 3 | 100 | 83.6 | 80.2 |
| Preparative Example 7 | 100 | 24.2 | 22.7 |
| Preparative Example 8 | 100 | 21.4 | 19.5 |
| Preparative Example 9 | 99 | 35.3 | 36.7 |

As can be seen from the results of Table 8, the compositions of Preparative Examples 1 to 9 showed significantly increased inhibitory activity against IL-17A and IL-23 compared to Comparative Examples 1 to 3, which do not contain octenidine dihydrochloride. The inhibitory activities of Preparative Examples 1, 2, and 3 increased by 2.5 to 4.6 times against IL-17A and 2.4 to 4.8 times against IL-23, compared to Comparative Example 1. The inhibitory activities of Preparative Examples 4, 5, and 6 increased by 2.9 to 4.4 times against IL-17A and 2.8 to 4.3 times against IL-23, compared to Comparative Example 2. The inhibitory activities of Preparative Examples 7, 8, and 9 increased by 2.4 to 3.9 times against IL-17A and 2.2 to 4.1 times against IL-23, compared to Comparative Example 3. Therefore, the pharmaceutical compositions of Preparative Examples 1 to 9 show good inhibitory activity against major cytokines (IL-17A, IL-23) inducing psoriasis, and thus can be expected to show excellent clinical therapeutic outcomes.

### (2) Cytotoxicity and Inhibitory Activity Against Inflammatory Cytokines in Mast Cells

Mast cells (human mast cell-1 (HMC-1)) were purchased from the Korean Cell Line Bank (Seoul, Korea) and used. The mast cells were cultured in IMDM (Iscove's Modified Dulbecco's Medium; Gibco BRL, USA) broth supplemented with 10% heat-inactivated fetal bovine serum (HIFBS; Gibco BRL, USA) and 1% penicillin/streptomycin (100 IU/ml penicillin, 100 μg/ml streptomycin; Thermo Fisher Scientific) for 24 hours in an incubator under conditions of 37 °C and 5% CO₂.

### \textcircled{1} Cytotoxicity Evaluation (MTT Assay)

100 μl of the cultured mast cells were seeded into a 96-well plate at 1×10⁶ cells/ml and cultured for 24 hours under conditions of 37 °C and 5% CO₂, and then the medium was removed, and 100 μl of DMEM medium and 100 μl of the samples (Comparative Examples 1 to 3, Preparative Examples 1 to 9) were added to each well and cultured for 24 hours. After 24 hours, 10 μl of 3-(4,5-dimethylthiazol-2-yl)-2,5-diphenyl tetrazolium bromide (MTT) solution (0.5 mg/ml, Sigma Aldrich, St. Louis, MO, USA) was added to each well and cultured for an additional 4 hours to reduce MTT, and then the supernatant was removed. Subsequently, 100 μl of DMSO was dispensed into each well and mixed for 1 hour, and then the absorbance was measured at 540 nm using an ELISA microplate reader (Bio-Rad model 680; Bio-Rad Laboratories INC., Tokyo, Japan). Cell viability was calculated as a percentage of the absorbance of the sample-treated group relative to the absorbance of the sample-untreated group, and the results are described in Table 9.

* Cell viability (%)=(Absorbance of sample-treated group/Absorbance of sample-untreated group)×100

### \textcircled{2} Inhibitory Activity Against Inflammatory Cytokines

The mast cells were seeded into a 96-well plate at a density of 5×10⁵ cells per well and cultured for 24 hours, and then 20 nM phorbol 12-myristate 13 acetate (PMA) and 1 μM calcium ionophore (calcium ionophore A23187) were added to each well, and simultaneously, treated with Comparative Examples 1 to 3 and Preparative Examples 1 to 9 at a concentration of 100 μg/ml each, and then cultured for 8 hours in an incubator under conditions of 37 °C and 5% CO₂. The culture was completed, and then the culture was centrifuged (2,000 rpm, 5 min) and the supernatant was collected, and to confirm the concentration of each cytokine (IL-4, IL-13) contained in the supernatant using an ELISA kit (LEGEND MAX; BioLegend, USA), the absorbance was measured at 450 nm with an ELISA microplate reader (Bio-Rad model 680, Bio-Rad Laboratories INC., Tokyo, Japan), and each cytokine inhibition concentration is described in Table 9.

TABLE 9

| | | Cytokine inhibitory | |
|---|---|---|---|
| | Cytotoxicity | activity (pg/ml) | |
| Samples | (cell viability %) | IL-4 | IL-13 |
| Comparative Example 1 | 100 | 2.9 | 5.2 |
| Preparative Example 1 | 100 | 0.9 | 1.6 |
| Preparative Example 2 | 100 | 0.7 | 1.2 |
| Preparative Example 3 | 101 | 1.0 | 2.0 |
| Comparative Example 2 | 100 | 2.5 | 7.6 |
| Preparative Example 4 | 101 | 0.8 | 2.4 |
| Preparative Example 5 | 101 | 0.6 | 2.0 |
| Preparative Example 6 | 99 | 1.0 | 2.7 |
| Comparative Example 3 | 100 | 2.7 | 6.2 |
| Preparative Example 7 | 100 | 0.9 | 1.9 |
| Preparative Example 8 | 99 | 0.7 | 1.5 |
| Preparative Example 9 | 101 | 1.1 | 2.6 |

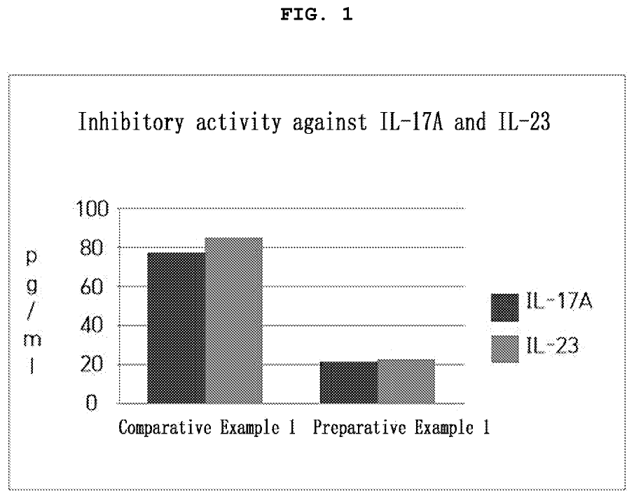

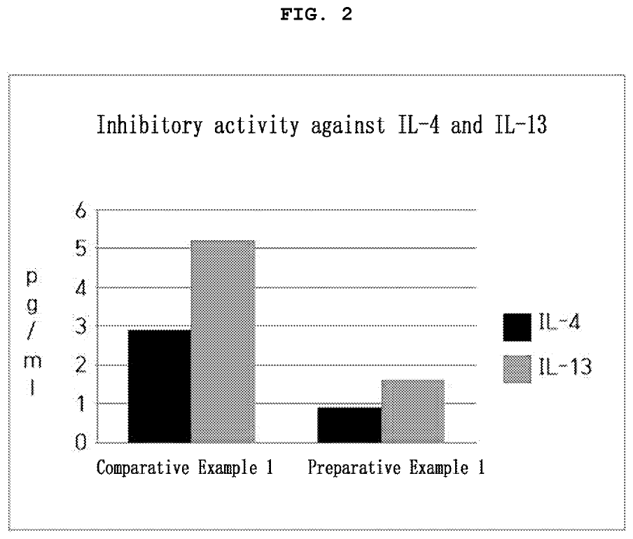

As can be seen from the results of Table 9, the compositions of Preparative Examples 1 to 9 showed significantly increased inhibitory activity against IL-4 and IL-13 compared to Comparative Examples 1 to 3, which do not contain octenidine dihydrochloride. The inhibitory activities of Preparative Examples 1, 2, and 3 increased by 2.9 to 4.1 times against IL-4 and 2.6 to 4.3 times against IL-13, compared to Comparative Example 1. The inhibitory activities of Preparative Examples 4, 5, and 6 increased by 2.5 to 4.2 times against IL-4 and 2.8 to 3.8 times against IL-13, compared to Comparative Example 2. The inhibitory activities of Preparative Examples 7, 8, and 9 increased by 2.5 to 3.9 times against IL-4 and 2.4 to 4.1 times against IL-13, compared to Comparative Example 3. Therefore, the pharmaceutical compositions of Preparative Examples 1 to 9 show good inhibitory activity against major cytokines (IL-4, IL-13) inducing atopic dermatitis, and thus can be expected to show excellent clinical therapeutic outcomes. FIG. 1 is a graph showing the experimental results of the inhibitory activities against IL-17A and IL-23 according to Comparative Example 1 and Preparative Example 1. Referring to FIG. 1, it can be seen that the composition according to Preparative Example 1 has superior inhibitory activity against IL-17A and IL-23 compared to Comparative Example 1. FIG. 2 is a graph showing the experimental results of the inhibitory activity against IL-4 and IL-13 according to Comparative Example 1 and Preparative Example 1. Referring to FIG. 2, it can be seen that the composition according to Preparative Example 1 has superior inhibitory activity against IL-4 and IL-13 compared to Comparative Example 1.

### Test Example 4. Evaluation Test of Crystal Precipitation Inhibitory Activity During Low-Temperature Storage

To evaluate crystal precipitation inhibitory activity during low-temperature storage, solutions containing a PDE-4 inhibitor and octenidine dihydrochloride were prepared according to the components and compositions in Tables 10, 11, and 12, as follows. Transparent solutions were prepared, by charging ethanol into preparation vessels at room temperature (25 °C), adding and dissolving roflumilast, difamilast, and tanimilast in each preparation vessel, and then sequentially adding octenidine dihydrochloride, polyethylene glycol 400, diisopropyl adipate, isopentyldiol, propylene carbonate, butylhydroxytoluene, disodium ethylenediaminetetraacetic acid, and citric acid to each preparation vessel, and adding purified water until the total weight of the solution reached 100% by weight, and then filtering them through a 0.45 μm filter. 10 ml of each prepared sample was placed into 20 ml vials and stored in a low-temperature freezer at −15 °C for 2 weeks, and then the state of crystal and precipitate formation of each sample was visually observed at room temperature (25 °C), and the results are described in Table 13.

TABLE 10

| | Preparative Examples (% by weight) | | | |
|---|---|---|---|---|
| | Comparative | Preparative | Preparative | Preparative |
| Components | Example 4 | Example 10 | Example 11 | Example 12 |
| Roflumilast | 0.50 | 0.50 | 0.50 | 0.50 |
| Octenidine dihydrochloride | 0.10 | 0.10 | 0.10 | 0.10 |
| Ethanol | 20.00 | 20.00 | 20.00 | 20.00 |
| Polyethylene glycol 400 | 10.00 | 10.00 | 10.00 | 10.00 |
| Diisopropyl adipate | 10.00 | 10.00 | 10.00 | 10.00 |
| Isopentyl diol | — | 10.00 | 20.00 | 30.00 |
| Propylene carbonate | — | 10.00 | 10.00 | 10.00 |
| Butylhydroxytoluene | 0.01 | 0.01 | 0.01 | 0.01 |
| Disodium ethylenediamine | 0.01 | 0.01 | 0.01 | 0.01 |
| tetraacetic acid | | | | |
| Citric acid | 0.10 | 0.10 | 0.10 | 0.10 |
| Purified water | 59.28 | 39.28 | 29.28 | 19.28 |
| Total (% by weight) | 100.00 | 100.00 | 100.00 | 100.00 |

TABLE 11

| | Preparative Examples (% by weight) | | | |
|---|---|---|---|---|
| | Comparative | Preparative | Preparative | Preparative |
| Components | Example 5 | Example 13 | Example 14 | Example 15 |
| Difamilast | 0.50 | 0.50 | 0.50 | 0.50 |
| Octenidine dihydrochloride | 0.10 | 0.10 | 0.10 | 0.10 |
| Ethanol | 20.00 | 20.00 | 20.00 | 20.00 |
| Polyethylene glycol 400 | 10.00 | 10.00 | 10.00 | 10.00 |
| Diisopropyl adipate | 10.00 | 10.00 | 10.00 | 10.00 |
| Isopentyl diol | — | 10.00 | 20.00 | 30.00 |
| Propylene carbonate | — | 10.00 | 10.00 | 10.00 |
| Butylhydroxytoluene | 0.01 | 0.01 | 0.01 | 0.01 |
| Disodium ethylenediamine | 0.01 | 0.01 | 0.01 | 0.01 |
| tetraacetic acid | | | | |
| Citric acid | 0.10 | 0.10 | 0.10 | 0.10 |
| Purified water | 59.28 | 39.28 | 29.28 | 19.28 |
| Total (% by weight) | 100.00 | 100.00 | 100.00 | 100.00 |

TABLE 12

| | Preparative Examples (% by weight) | | | |
|---|---|---|---|---|
| | Comparative | Preparative | Preparative | Preparative |
| Components | Example 6 | Example 16 | Example 17 | Example 18 |
| Tanimilast | 0.50 | 0.50 | 0.50 | 0.50 |
| Octenidine dihydrochloride | 0.10 | 0.10 | 0.10 | 0.10 |
| Ethanol | 20.00 | 20.00 | 20.00 | 20.00 |
| Polyethylene glycol 400 | 10.00 | 10.00 | 10.00 | 10.00 |
| Diisopropyl adipate | 10.00 | 10.00 | 10.00 | 10.00 |
| Isopentyl diol | — | 10.00 | 20.00 | 30.00 |
| Propylene carbonate | — | 10.00 | 10.00 | 10.00 |
| Butylhydroxytoluene | 0.01 | 0.01 | 0.01 | 0.01 |
| Disodium ethylenediamine | 0.01 | 0.01 | 0.01 | 0.01 |
| tetraacetic acid | | | | |
| Citric acid | 0.10 | 0.10 | 0.10 | 0.10 |
| Purified water | 59.28 | 39.28 | 29.28 | 19.28 |
| Total (% by weight) | 100.00 | 100.00 | 100.00 | 100.00 |

TABLE 13

| Comparative | Preparative | Preparative | Preparative |
|---|---|---|---|
| Example 4 | Example 10 | Example 11 | Example 12 |
| Crystals/precipitates | Crystals/precipitates | Crystals/precipitates | Crystals/precipitates |
| → present | → present | → absent | → present |
| Comparative | Preparative | Preparative | Preparative |
| Example 5 | Example 13 | Example 14 | Example 15 |
| Crystals/precipitates | Crystals/precipitates | Crystals/precipitates | Crystals/precipitates |
| → present | → present | → absent | → present |
| Comparative | Preparative | Preparative | Preparative |
| Example 6 | Example 16 | Example 17 | Example 18 |
| Crystals/precipitates | Crystals/precipitates | Crystals/precipitates | Crystals/precipitates |
| → present | → present | → absent | → present |

Referring to the results in Table 13, it was confirmed that in the cases of Preparative Examples 11, 14, and 17, where the weight ratio of isopentyl diol to propylene carbonate as the crystal growth inhibitor was 2:1 and the content of the crystal growth inhibitor was 30% by weight based on the total weight of the composition, no crystal precipitation occurred even after storage at −15 °C for 2 weeks. As described above, according to various embodiments of the present invention, it not only has excellent preventive and therapeutic effects on psoriasis and atopic dermatitis but also has high medication compliance due to its excellent microbicidal effects. In addition, according to various embodiments of the present invention, the composition of the present invention exhibits much superior microbicidal effects compared to conventional single-component formulations of PDE-4 inhibitors. That is, the pharmaceutical composition according to various embodiments of the present invention has excellent microbicidal effects against Gram-positive bacteria, yeasts, fungi, and the like.

In addition, according to various embodiments of the present invention, it preserves formulations in good condition, exhibits excellent microbicidal effects on inflammatory areas, and demonstrates improved inhibitory activity against inflammatory cytokines.

Furthermore, according to various embodiments of the present invention, the pharmaceutical composition has excellent storage stability even when stored at 45 °C for 4 weeks.

As described above, although the specific description of the present invention has been made through embodiments, the aforementioned embodiments have been described only as preferred examples of the present invention, and therefore, it should not be understood that the present invention is limited only to the aforementioned embodiments, and the scope of rights of the present invention should be understood as the following claims and equivalent concepts thereof.

What is claimed is:

1. A pharmaceutical composition for preventing or treating psoriasis and atopic dermatitis, comprising:
a phosphodiesterase-4 inhibitor comprising at least any one of a phosphodiesterase-4 inhibitory component and a pharmaceutically acceptable salt thereof;
an active ingredient comprising at least any one of octenidine and an octenidine salt; and
a crystal growth inhibitor comprising isopentyldiol and propylene carbonate in a weight ratio of 2:1,
wherein the phosphodiesterase-4 inhibitory component comprises at least any one of roflumilast, difamilast, and tanimilast, and
characterized in that the crystal growth inhibitor has a content of 1 to 30% by weight based on the total weight of the composition.

2. The pharmaceutical composition for preventing for treating psoriasis and atopic dermatitis according to claim 1, characterized in that the phosphodiesterase-4 inhibitor has a content of 0.01 to 5% by weight based on the total weight of the composition.

3. The pharmaceutical composition for preventing or treating psoriasis and atopic dermatitis according to claim 1, characterized in that the phosphodiesterase-4 inhibitory component further comprises at least any one of roflumilast N-oxide, lotamilast, apremilast, orismilast, crisaborole, and rolipram.

4. The pharmaceutical composition for preventing or treating psoriasis and atopic dermatitis according to claim 1, characterized in that the pharmaceutically acceptable salt of the phosphodiesterase-4 inhibitory component includes at least any one selected from an inorganic ion salt, an inorganic acid salt, an organic acid salt, a sulfonic acid salt, an amino acid salt, and an amine salt of the phosphodiesterase-4 inhibitory component.

5. The pharmaceutical composition for preventing or treating psoriasis and atopic dermatitis according to claim 1, characterized in that:
the active ingredient has a content of 0.01 to 8% by weight based on the total weight of the composition, and
the octenidine salt includes at least any one selected from an inorganic ion salt, an inorganic acid salt, an organic acid salt, a sulfonic acid salt, an amino acid salt, and an amine salt of the octenidine.

6. The pharmaceutical composition for preventing or treating psoriasis and atopic dermatitis according to claim 1, characterized in that the pharmaceutical composition for preventing or treating psoriasis and atopic dermatitis further comprises:
an additive including at least any one of a solvent, a humectant, a penetrant, a surfactant, an antioxidant, a chelating agent, a pH adjuster, and a viscosity modifier.

7. The pharmaceutical composition for preventing or treating psoriasis and atopic dermatitis according to claim 1, characterized in that the pharmaceutical composition for preventing or treating psoriasis and atopic dermatitis has a pH of 2 to 10.

8. The pharmaceutical composition for preventing or treating psoriasis and atopic dermatitis according to claim 1, characterized in that the pharmaceutical composition for preventing or treating psoriasis and atopic dermatitis has a density of 0.1 to 2 g/cm³.

9. The pharmaceutical composition for preventing or treating psoriasis and atopic dermatitis according to claim 1, characterized in that the pharmaceutical composition for preventing or treating psoriasis and atopic dermatitis has a surface tension of 1 to 75 dyn/cm.

10. The pharmaceutical composition for preventing or treating psoriasis and atopic dermatitis according to claim 1, characterized in that the pharmaceutical composition for preventing or treating psoriasis and atopic dermatitis has a moisture content of 0.1 to 95% by weight.

11. The pharmaceutical composition for preventing or treating psoriasis and atopic dermatitis according to claim 1, characterized in that the pharmaceutical composition for preventing or treating psoriasis and atopic dermatitis has any one formulation selected from a solution, an ointment, a lotion, a cream, a gel, an emulsion, a suspension, a stick, a plaster, a patch, a cataplasma, a microcapsule, a cleaning agent, a liposomal drug, and a spray.
